(19) United States
(12) Patent Application Publication
(10) Pub. No.: US 2013/0172987 A1
(43) Pub. Date: Jul. 4, 2013

(54) MEDICAL DEVICE COMPRISING A POROUS ARTICLE OF EPTFE EXHIBITING IMPROVED CELLULAR TISSUE INGROWTH

(71) Applicants: Helmut Goldmann, Kassel (DE); Dennis Langanke, Tübingen (DE)

(72) Inventors: Helmut Goldmann, Kassel (DE); Dennis Langanke, Tübingen (DE)

(73) Assignee: Aesculap AG, Tuttlingen/Donau (DE)

(21) Appl. No.: 13/578,022

(22) PCT Filed: Feb. 11, 2011
(86) PCT No.: PCT/EP11/52032

§ 371 Date: Oct. 31, 2012

(30) Foreign Application Priority Data
Feb. 12, 2010 (EP) 10001486.9

(51) Int. Cl. A61F 2/82

(52) U.S. Cl. CPC A61F 2/82
USPC 623/1.46; 427/2.25

(57) ABSTRACT

A medical device comprising a porous article of expanded polytetrafluoroethylene (ePTFE) having a microstructure comprising nodes interconnected by fibrils, wherein the microstructure at a surface of the article at least partially includes nodes having segments free of fibrillate interconnections.

### RELATED APPLICATIONS

This is a §371 of International Application No. PCT/EP2011/052032, with an international filing date of Feb. 11, 2011 (WO 2011/098558 A1, published Aug. 18, 2011), which is based on European Patent Application No. 10001486.9, filed Feb. 12, 2012, the subject matter of which is incorporated by reference.

### TECHNICAL FIELD

This disclosure relates generally to a medical device comprising a porous article of expanded polytetrafluoroethylene (ePTFE) having a microstructure at the surface of the article promoting tissue ingrowth and a method for manufacturing the medical device.

### BACKGROUND

Generally, vascular prostheses should have an inside surface that is as smooth as possible to avoid platelet activation and adhesion that might lead to deposit of fibrin layers on the inside surface, thereby causing occlusion of the prostheses. Besides, vascular prostheses should have an outside surface that facilitates tissue ingrowth to provide a secure anchoring of the prosthesis with surrounding tissues of a human or animal body.

It is already known that fibroblasts readily enter a vascular prosthesis made of knitted or woven fabric based on polyester that is due to the loose structure of the tubular wall of such a prosthesis. However, knitted or woven prostheses generally suffer from the drawback that bleeding occurs through the wall immediately after implantation which results in undesired platelet activation and adhesion finally leading to occlusion of the prostheses. Besides, blood trapped by the prosthesis structure may cause the manifestation of seroma normally associated by an inflammatory response. Thus, macrophages may be attracted that may lead to a degradation of the prosthesis.

Further, it is known to use vascular prostheses made of expanded polytetrafluoroethylene (ePTFE) having a porous microstructure defined by nodes interconnected by fibrils. Known ePTFE prostheses normally have a pore structure at the inside surface that satisfactorily lowers the risk of platelet activation and subsequent adhesion on the inside surface. However, due to the above mentioned node and fibril microstructure of ePTFE the entry of fibroblasts into the prostheses is generally deficient and normally lasts over a long period of time. Accordingly, several attempts were made to optimize the ingrowth of fibroblast into ePTFE prostheses. For instance, reference is made to U.S. Pat. No. 4,208,745, U.S. Pat. No. 4,877,661, U.S. Pat. No. 4,713,070 and the U.S. Pat. No. 5,433,909. However, often laborious and time-consuming techniques are necessary without significantly improving cellular entry into ePTFE prostheses.

Accordingly, it could be helpful to provide a medical device made of ePTFE having a microstructure at its surface facilitating an easy natural tissue ingrowth from the outer periphery of the medical device that is preferably comparable to the tissue ingrowth characteristics of known knitted or woven vascular grafts without increasing the risk for the patient.

### SUMMARY

We provide a medical device including a porous article of expanded polytetrafluoroethylene (ePTFE) having a microstructure including nodes interconnected by fibrils, wherein the microstructure at a surface of the article at least partially includes nodes having segments free of fibrillate interconnections.

We also provide a method of manufacturing the medical device, including subjecting a porous article of expanded polytetrafluoroethylene (ePTFE) having a microstructure of nodes interconnected by fibrils to a surface treatment forming nodes having segments free of fibrillate interconnections at a surface of the article.

### BRIEF DESCRIPTION OF THE DRAWINGS

FIG. 1 is a schematic illustration of the node and fibril microstructure of conventional ePTFE.

FIG. 2 is a schematic illustration of our node and fibril microstructure of ePTFE.

FIG. 3 is a REM photo of the longitudinal cut of our ePTFE vascular prosthesis.

### DETAILED DESCRIPTION

Our medical device comprises a porous article of expanded polytetrafluoroethylene (ePTFE) having a microstructure comprising interspaced nodes interconnected by fibrils, wherein the microstructure at a surface of the article, preferably at an outside surface thereof, at least partially comprises nodes having segments (node segments) free of fibrillate interconnections (cross-links). Preferably, the nodes having segments being free of fibrillate interconnections are only present at the surface, in particular an outside surface, of the porous article.

Conventional ePTFE prostheses are featured by a microfibrous structure composed of interspaced nodes interconnected by fibrils. Our medical device differs from conventional prostheses in that its microstructure at the surface of the article, preferably at an outside surface thereof, includes node segments not interconnected by fibrils (i.e., are free of fibrillate interconnections). In other words, the surface of the porous article, preferably an outside surface thereof, is free of fibrillate interconnections. Thus, the corresponding internodal spaces are restricted to a lesser extent by fibrillate interconnections. This leads to manifestation of enlarged cavities or openings, in particular pores, at the surface of the article, facilitating an easier tissue entry into the article from its outer periphery. This is favorable in secure anchoring of the device. Furthermore, the healing process after implantation of the medical device is supported by an easier tissue entry into the device.

The term “polytetrafluoroethylene” as used herein also comprises a copolymer of tetrafluoroethylene.

Accordingly, the term “copolymer” as used herein preferably means a polymer that comprises at least two different monomer units.

The term “copolymer of tetrafluoroethylene” as used herein encompasses a polymer composed of at least one further monomer unit along tetrafluoroethylene. Such a further monomer unit may be selected from the group consisting of chlorotrifluoroethylene, perfluoroalkoxytetrafluoroethylene, hexafluoropropylene, tetrafluoropropylene and combinations thereof.

Preferably, the node segments being free of fibrillate interconnections are aligned (oriented) to the surface of the article. In particular, the node segments free of fibrillate interconnections form an outer periphery of the porous article.

Preferably, the node segments that are free of fibrillate interconnections protrude, preferably pillar-like, from the surface, in particular from an outside surface, of the porous article. Accordingly, these node segments, effect a roughly structured surface that promotes tissue entry, typically connective tissue entry, from the outer periphery of the article. Particularly, the node segments being free of fibrillate interconnections facilitating the trapping of body cells, in particular of fibroblasts, triggering cellular entry into the article. More preferably, the node segments that are free of fibrillate interconnections confer the surface of the article a velour-like appearance.

The nodes at the surface of the porous article may be predominantly free of fibrillate interconnections. Particularly, the node segments being free of fibrillate interconnections have a length of 1 to 99%, in particular 2 to 80%, preferably 5 to 70%, more preferably 10 to 45%, and especially preferably 15 to 35%, in relation to the total length of the nodes. Preferably, the node segments being free of fibrillate interconnections have a length of 1.5 to 795 μm, in particular 3 to 600 μm, preferably 5 to 400 μm, more preferably 10 to 250 μm and especially preferably 20 to 150 μm.

Preferably, basically all nodes, in particular all nodes, at the surface of the article comprise segments free of fibrillate interconnections. Preferably, the node segments that are free of fibrillate interconnections are arranged in the form of a zone (area or sphere) at the surface of the porous article having a proportion of 2 to 30%, in particular 5 to 20%, and preferably 10 to 15%, relating to the thickness, in particular wall thickness, of the porous article. This has the advantage, that body cells, in particular fibroblasts, cannot infiltrate the porous article in its entirety which might cause an undesired passage of the cells into a lumen of the porous article causing an undesired occlusion thereof. Preferably, the zone has a thickness of 4 to 300 μm, in particular 20 to 150 μm, particularly 20 to 80 μm, and preferably 40 to 80 μm. Further preferably, the zone may have a thickness of 20 to 25 μm.

The node segments that are free of fibrillate interconnections may have fibrillate protrusions. The protrusions may extend from the body of the nodes and node segments, respectively over a length of 1 to 150 μm, in particular 10 to 100 μm, and preferably 20 to 80 μm. Normally, the protrusions have a length less than the length of the interconnecting fibrils and/or internodal spaces of the microstructure. Accordingly, the protrusions normally offer a significantly reduced resistance to cellular ingrowth in comparison to fibrils that interconnect nodes.

As already mentioned, the microstructure is based on nodes interconnected by fibrils. Preferably, nodes being along their size, in particular length, preferably continuously interconnected by fibrils may be arranged in the form of a zone (area or sphere) having a proportion of 98 to 70%, in particular 95 to 80%, and preferably 90 to 85%, taken at the thickness, in particular wall thickness, of the porous article. The zone may have a thickness of 140 to 980 μm, in particular 250 to 750 μm, and preferably 425 to 475 μm. It is especially preferred that the zone of nodes being along their size, in particular length, preferably continuously interconnected by fibrils is beneath a zone of nodes having segments free of fibrillate interconnections. Further preferably, the nodes being along their size, in particular length, continuously interconnected by fibrils are present at an inside surface of the porous article.

Preferably, the nodes of the microstructure have an elongate, in particular an ellipsoidal, shape. More preferably, the nodes have a relatively uniform shape and in particular a uniform size. The nodular size, particularly the nodular length, may to 1 to 800 μm, in particular 5 to 500 μm, and preferably 10 to 300 μm. The interconnecting fibrils may have a bent, wavy or straight appearance. More preferably, fibrils that interconnect nodes have a straight and parallel appearance. Further, the fibrils may have a length of 1 to 150 μm, in particular 20 to 80 μm. Furthermore, the fibrils may have a diameter of 0.1 to 10 μm, in particular 0.5 to 5 μm. The nodes of the microstructure may essentially be oriented in a direction perpendicular to the longitudinal and lateral direction of the porous article.

The microstructure may include nodes arranged in differing internodal spaces (spaces between the nodes). Preferably, the internodal spaces are arranged in a gradient. More preferably, internodal spaces increase, in particular gradually increase, from one surface to an opposing surface of the porous article. Typically, the nodes of the microstructure may have an internodal space of 1 to 150 μm, in particular 10 to 100 μm, and preferably 20 to 80 μm.

The porous article may comprise a coating, preferably including an antimicrobial material, in particular selected from the group consisting of an antimicrobial metal, antimicrobial alloy, antimicrobial compound, antimicrobial salt and combinations thereof. Antimicrobial metals, antimicrobial alloys and/or antimicrobial metal compounds, in particular antimicrobial metal salts, are especially preferred. Typically, only the surface of the porous article, preferably only a part thereof, comprises the coating. More preferably, the node segments free of fibrillate interconnections are at least partially, in particular completely, coated, preferably with an antimicrobial material. In particular, only the node segments being free of fibrillate interconnections are at least partially, in particular completely, coated, preferably with an antimicrobial material. The remaining segments of these nodes are preferably interconnected by fibrils, i.e., comprise fibrillate interconnections. The remaining segments are in particular aligned (oriented) in a direction converse to an outside surface of the porous article.

Further, it is preferred that internodal spaces at the surface of the porous article are free of a coating, particularly free of a coating including an antimicrobial material. In other words, the coating preferably leaves the internodal spaces open at the surface of the porous article.

A suitable antimicrobial material may be selected from the group consisting of zirconium, copper, zinc, silver, gold, palladium, platinum, iridium, aluminum, nickel, tungsten, molybdenum, tantalum, titanium, iodine, alloys thereof, compounds, in particular salts, for instance oxides, thereof and combinations thereof.

As used herein, the term “antimicrobial material” preferably refers to a material which inhibits the growth of microorganisms such as pathogenic bacteria, protists and/or fungi, which can cause infections within a patient.

Further, the coating, in particular including an antimicrobial material, may have a layer thickness of 100 to 4000 Å, in particular 400 to 2500 Å, preferably 800 to 1600 Å.

Furthermore, the coating may have a proportion of 0.01 to 5.0% by weight, in particular 0.1 to 3.0% by weight, and preferably 0.3 to 1.5% by weight, relating to the total weight of the porous article.

Preferably, the coating is applied to the porous article in a depth of 1 to 150 μm, in particular 20 to 100 μm, particularly 20 to 80 μm, and preferably 40 to 80 μm, taken at the surface, preferably taken at an outside surface, of the porous article. Further preferably, the coating is applied to the porous article in a depth of 20 to 25 μm.

The porous article may comprise an adhesion-promoting agent. Typically, the adhesion-promoting agent is adjacent to the surface of the article. Preferably, the adhesion-promoting agent is arranged in the form of a layer. Further, the adhesion-promoting agent may be a metal, in particular titanium. The porous article may further comprise a species, in particular a layer thereof, which forms a barrier to degradation or diffusion of an adhesion-promoting agent and, for instance, impede galvanic interaction between an antimicrobial material and an adhesion-promoting agent. The species may be a metal, in particular palladium. Preferably, the barrier species is adjacent to the surface of the porous article and in particular interposed between an adhesion-promoting agent and an antimicrobial material.

The porous article may comprise a leak-proofing coating or impregnation. Such a coating and impregnation, respectively, is typically designed on the surface of the article. Suitable materials for the coating and impregnation, respectively, may be biopolymers and/or synthetic polymers, for example, copolymers. Suitable materials may be selected from the group consisting of collagen, gelatin, albumin, polyvinylalcohol, carboxymethylcellulose and combinations thereof. Further suitable materials may be selected from the group consisting of polylactide, polyglycolide, poly-ε-caprolactone, polytrimethylencarbonate, poly-para-dioxanone, copolymers thereof and combinations thereof. Typically, such a coating and impregnation, respectively, may seal internodal spaces at the surface of the article. Due to the node segments that are free of fibrillate interconnections the afore-described coating and impregnation, respectively, may more strongly adhere to the surface of the article.

The porous article may be free of a leakproofing coating or impregnation, in particular as described above.

Preferably, the node segments free of fibrillate interconnections result from a surface treatment, in particular from a dry coating process, preferably from a physical vapor deposition process (PVD process), more preferably from an ion-beam-assisted deposition process (IBAD process), in particular of an antimicrobial material, preferably of an antimicrobial metal. More details thereof are described in the following description.

Preferably, the medical device is a surgical implant.

Especially preferably, the porous article is a tubular article, typically a hollow tubular article. In other words, the porous article is preferably designed as a tubing, typically having a lumen encased by a tubing wall. More preferably, the node segments free of fibrillate interconnections are present at the outside (exterior) surface of the tubular article. Further, it is preferred that nodes being along their size, in particular length, preferably continuously interconnected by fibrils are present at the inside (interior) surface of the tubular article. Pores, in particular internodal spaces, at the outside surface of the tubular article may differ from those at the inside surface of the tubular article. It is especially preferred that the inside surface of the tubular article is essentially arranged smooth. In other words, the microstructure at the inside surface of the tubular article preferably offers no or only a negligible resistance to flow of blood and, consequently, platelet adhesion may be reduced. Furthermore, it is preferred that the inside surface may have a pore structure, in particular internodal spaces, that facilitates the passage of small molecular compounds, particularly of nutrients, biological agents and/or medical agents through the wall of the tubular article into the lumen thereof. Thus, a nutrition supply to a neointima is possible that preferably lines the inside surface of the tubular article. It is thus possible to greatly reduce calcification of the neointima that may result from nutritional deficiency.

Further, biological and/or medical agents may enter the lumen of the tubular article and, for instance, may help to prevent the manifestation of a thrombosis. Preferably, the microstructure at the outside surface of the tubular article comprises a pore structure, in particular internodal spaces, that promotes tissue ingrowth, in particular ingrowth of fibroblasts, from the outer periphery of the article. This contributes to a secure anchoring of the article with surrounding connective tissue. Furthermore, the aforementioned nutrient supply is essentially based on capillaries which densely develop on fully grown fibroblasts. Preferably, pore sizes, in particular internodal spaces, of the microstructure increase, particularly gradually increase, from the inside surface to the outside surface of the tubular article.

The porous article may be designed as a tubular article having an internal diameter of 2 to 50 mm.

Further, the porous article may have a thickness, in particular wall thickness, of 0.1 to 1.0 mm, in particular 0.25 to 0.75 mm, preferably 0.4 to 0.6 mm.

Alternatively, the porous article is designed as a two-dimensionally shaped (planar) article. Preferably, the node segments being free of fibrillate interconnections are present only at one surface of the two-dimensional shaped article, in particular facilitating cellular ingrowth into the article from the one surface. The opposing surface preferably comprises a microstructure based on nodes being along their size, in particular length, continuously interconnected by fibrils, in particular preventing cellular ingrowth from the opposing surface and preferably avoiding post-surgical adhesion with the opposing surface. The porous article may be designed as a mesh such as a surgical mesh, in particular for the repair of hernias, or as a patch, in particular for hemostasis.

Further preferably, the medical device is selected from the group consisting of tubular prostheses, catheters, stents, shunts, hernia meshes, prolaps meshes, and aconuresis slings. More preferably, the medical device is a tubular prosthesis, in particular a vascular prosthesis (vascular graft). More specifically, the medical device may be an arterial prosthesis or a vein prosthesis. However, it is especially preferred that the medical device is an arterial prosthesis.

Further, the medical device may be on hand in a sterile form and in particular tailored form.

We further provide a method for the manufacture of a medical device comprising the step of subjecting a porous article of expanded polytetrafluoroethylene (ePTFE) having a microstructure of interspaced nodes interconnected by fibrils to a surface treatment, forming nodes having segments free of fibrillate interconnections at a surface of the porous article, preferably at an outside surface thereof.

Typically, the porous article is provided as a tubular article, i.e., tubing, having an inside surface and an outside surface. It is especially preferred that only the outside surface of the tubular article is subjected to the surface treatment. Thus, the node segments being free of fibrillate interconnections are preferably formed at the outside surface of the tubular article. The microstructure at the inside surface of the tubular article is preferably not affected by the surface treatment.

Preferably, the fibrils interconnecting the nodes are at least partially, in particular completely, open broken, disintegrated, in particular melted on, by the surface treatment. The microstructure at the surface of the article may at least partially be, in particular completely, altered or modified by the surface treatment.

Preferably, the surface treatment additionally includes the introduction, in particular deposition, of an antimicrobial material, in particular an antimicrobial metal, at the surface of the porous article. Thus, a porous article may be produced in one step having a microstructure at the surface thereof facilitating an easier tissue ingrowth and preferably having a long-lasting antimicrobial protection.

Preferably, the surface treatment is performed by a dry coating process, in particular by a physical vapor deposition process (PVD process). More preferably, the surface treatment is performed by an ion-beam-assisted deposition process (IBAD process). In general, ion-beam processes are low-temperature, high-technology processes with excellent quality control to achieve good adherence, ductility, reproducibility, reliability and thickness of deposition control at a high throughput and with no chemical residues, thus being both environmentally and occupationally a safe dependable technique. Typically, an ion-beam-assisted coating apparatus comprises a vacuum chamber system formed of a low-vacuum antechamber and a high vacuum processing chamber, air-tightly separated from each other by a gate movable between an opened position and a closed position. An ion source, which can be a bucket type ion source is mounted within the high-vacuum processing chamber, normally in a position diametrically opposed to the low-vacuum antechamber. The ion source is typically fed by one or more gases such as argon, oxygen, neon, and/or helium, from a suitable gas supply source via a mess flow controller, regulating the rate of gas feed.

Further, an evaporator is also mounted in the high-vacuum processing chamber, normally in operative association with the ion source. The evaporator is designed to vaporize particular evaporants, more specifically metallic evaporants to dry-coat a specific substrate, in particular a medical device, therewith, being assisted in the dry-coating by an ion-beam emanating from the ion source. Suitable evaporants include zirconium, copper, zinc, silver, gold, palladium, platinum, iridium, aluminum, nickel, tungsten, molybdenum, tantalum, titan and their respective alloys, oxides and compounds. Normally, a vapor shutter, designed to be rotated in and out of place of the evaporator, shields the substrates from the evaporants when in place. The substrate to be dry-coated is normally introduced into the vacuum chamber system with the aid of a suitable substrate holder. Preferably, the substrate holder is mounted for both rotational and translatory motion on a shaft and introduced in the antechamber through a hinge-like mounted end-plate.

The IBAD process is typically performed in a suitable vacuum chamber system including a processing chamber. The required vacuum environment is normally created by a vacuum pump. Preferably, the ion-beam-assisted deposition is performed under a vacuum pressure of at least 10-4 torr, particularly at least 10-5 torr. Further, the ion-beam-assisted deposition may be performed at a temperature <150° C. Moreover, the porous article may be exposed to an ion-beam energy of 5 to 10000 eV, in particular 50 to 5000 eV, preferably 200 to 1000 eV. The above mentioned ion-beam energy is preferably intended to achieve an ion beam current density on the surface of the porous article of 0.1 to 500 μA/cm2, in particular 1 to 250 μA/cm2, preferably 10 to 80 μA/cm2. The ion-beam-assisted deposition process may be performed during a time period of 0.1 to 500 minutes, in particular 1 to 200 minutes, preferably 5 to 75 minutes. Further, the ion-beam-assisted deposition process may be performed applying a deposition rate of 0.1 to 50 Å(angstroms)/second, in particular 1 to 25 Å(angstroms)/second, preferably 2 to 10 Å(angstroms)/second.

Particularly preferably, the ion-beam-assisted deposition is carried out under the following process parameters: a vacuum pressure of at least 10-5 torr, an ion beam energy of 200 eV to 1000 eV, an ion beam current density of 10 to 80 μA/cm2 and a deposition rate of 2 to 10 Å(Angstroms)/second.

The surface treatment may be performed applying a mechanical method, in particular brushing. Alternatively, the surface treatment is performed applying a thermal method, in particular employing laser techniques. Furthermore, sputtering or plasma treatment may be employed.

We also provide a medical device, preferably a vascular prosthesis, obtained or obtainable according to one of the afore-described methods. For further details and advantages, reference is made to the previous description.

Due to its specific microstructure, the device may be more quickly integrated into environmental tissue within a patient's body. Advantageously, four weeks subsequent to implantation, the medical device is featured by vascularization which is three-to-five-fold more distinct in comparison to medical devices having a conventional ePTFE microstructure. As used herein, the term “vascularization” preferably relates to the formation and ingrowth, respectively, of blood vessels into a newly formed tissue. Further, blood vessels which have been grown into the device four weeks after its implantation, preferably have a capillary cross-section of 4 and 40 mm2, in particular 6 and 30 mm2, and preferably 8 and 20 mm2.

Due to the specific microstructure of the medical device, a connective tissue-like organization on the surface, in particular on an outside surface thereof, is also more quickly induced. For instance, four weeks after implantation of the device, a more distinct formation of a periimplantat capsule and of a periimplantat fibrosis is visible. The periimplantat capsule may have a thickness after four weeks from implantation which is 30 and 130 μm, in particular 50 and 110 μm, and preferably 60 and 100 μm.

Our devices and methods will be illustrated in more detail by a disclosure of preferred examples presented in the figures and a description of the figures. Individual features may be realized exclusively or in combination with other features. Any described example is given for the sole purpose of illustration and better understanding, and is no way to be interpreted as a limitation.

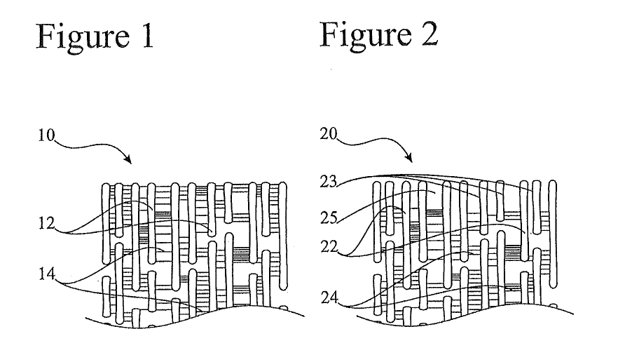

FIG. 1 schematically illustrates a known node fibril microstructure 10 of ePTFE realized in conventional ePTFE prostheses. The microstructure 10 comprises interspaced nodes 12 that have typically an elongate shape and are interconnected by fibrils 14. Importantly, also the nodes 12 being present at the surface 11 of the microstructure 10 are interconnected by fibrils 14. Dependent on the stretching and expanding conditions during the manufacture of ePTFE the form of the nodes 12 and in particular the appearance of the interconnecting fibrils 14 may vary. For example, the fibrils 14 may have a bent, wavy or, as illustrated in FIG. 1, a straight and in particular parallel-appearance.

FIG. 2 schematically illustrates the microstructure 20 based on nodes 22 interconnected by fibrils 24 in our medical device. Accordingly, the microstructure 20 comprises at the surface 21, preferably at an outside surface, of the device nodes 22 having segments 23 free of fibrillate interconnections 24. In other words, internodal spaces 25 are present at the surface 21 of the device that are not restricted by fibrillate interconnections 24. Thus, the medical device comprises enlarged openings or cavities, in particular pores, at its surface 21 allowing for an easier entry of connective tissue cells, in particular fibroblasts, into the device from its outer periphery. Thus, recovery of the original present anatomical situation in the body of a patient before implanting the device is accelerated. Furthermore, an easier entry of connective tissue cells into the device contributes to a secure anchoring of the device in the body of a patient. Preferably, the node segments 23 are coated with an antimicrobial material.

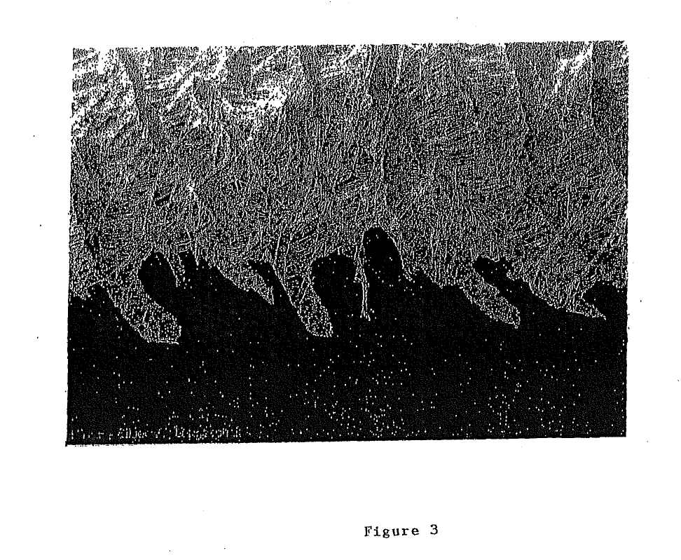

FIG. 3 displays a REM picture of the longitudinal cut of our ePTFE vascular prosthesis. The REM picture clearly shows that the nodes at the outside surface of the vascular prosthesis comprise segments completely free of fibrillate interconnections forming enlarged cavities or openings at the outside surface of the prosthesis. The node segments preferably protrude from the outside surface and in particular confer the outside surface a more roughly structure, in particular a velour-like appearance. The specific microstructure at the outside surface of the prosthesis preferably depending on the aforementioned features facilitates the entry of body cells, in particular connective tissue cells, preferably fibroblasts, from the outer periphery of the vascular prosthesis. This leads to a secure anchoring of the prosthesis within the body of a patient and to a natural recovery of the implantation region.

### EXAMPLE

Prostheses of ePTFE are clamped in a rotatable clamp device so that they hang freely as a bundle of parallel tubes with spaces between them. The clamp device is introduced into a vacuum chamber suitable for carrying out the IBAD technique, the ePTFE prostheses being vapor-deposited with silver and at the same time bombarded with argon ions. The coating operation is conducted until a silver layer thickness of 1300 Å is reached on the outside surface of the ePTFE prosthesis or the fibrils located there. If desired, a primary coating of other metals can be effected by vapor-deposition.

An ePTFE prosthesis with an internal diameter of 8 mm and a wall thickness of 500 μm coated in the above-described way has node segments at the outside surface which are free of fibrillate interconnections. The node segments being free of fibrillate interconnections form a zone having a layer thickness of 40 to 80 μm. The microstructure that is based on nodes interconnected by fibrils has a layer thickness of 420 to 460 μm. The silver coating typically have a film thickness of about 1300 Åresulting in a proportion of silver relative to the total weight of 0.3 to 0.5% by weight. The coating is applied to the ePTFE prosthesis in a depth of 40 to 80 μm.

1. A medical device comprising a porous article of expanded polytetrafluoroethylene (ePTFE) having a microstructure comprising nodes interconnected by fibrils, wherein the microstructure at a surface of the article at least partially comprises nodes having segments free of fibrillate interconnections.

2. The medical device according to claim 1, wherein the node segments are aligned to the surface of the article, form an outer periphery of the article.

3. The medical device according to claim 1, wherein the node segments protrude from the surface of the porous article.

4. The medical device according to claim 1, wherein the node segments have a length of 1 to 99% relative to total length of the nodes.

5. The medical device according to claim 1, wherein the node segments are arranged in a zone having a proportion of 2 to 30% taken at a wall thickness of the porous article.

6. The medical device according to claim 1, wherein the surface of the porous article comprises a coating including an antimicrobial material.

7. The medical device according to claim 1, wherein the node segments are at least partially coated with an antimicrobial material.

8. The medical device according to claim 6, wherein the coating including an antimicrobial material leaves internodal spaces open at the surface of the porous article.

9. The medical device according to claim 6, wherein the coating has a layer thickness of 100 to 4000 Å.

10. The medical device according to claim 6, wherein the coating is applied to the porous article at a depth of 1 to 150 μm taken at the surface of the porous article.

11. The medical device according to claim 1, wherein the porous article is a tubular article and the node segments are present at an outside surface of the tubular article.

12. The medical device according to claim 1, which is a tubular prosthesis.

13. A method of manufacturing a medical device according to claim 1, comprising subjecting a porous article of expanded polytetraflouroethylene (ePTFE) having a microstructure of nodes interconnected by fibrils to a surface treatment forming nodes having segments free of fibrillate interconnections at a surface of the article.

14. The method according to claim 13, wherein the surface treatment is performed by a physical vapor deposition process (PVD process) or an ion-beam-assisted deposition process (IBAD process).

15. (canceled)
